(19) United States
(12) Patent Application Publication
(10) Pub. No.: US 2026/0054105 A1
(43) Pub. Date: Feb. 26, 2026

(54) WEARABLE AIR PURIFIER

(71) Applicant: Dyson Technology Limited, Malmesbury, Wiltshire (GB)

(72) Inventors: Benjamin James HOVELL, Tetbury (GB); Michael John COLLISON, Swindon (GB)

(21) Appl. No.: 19/099,598

(22) PCT Filed: Aug. 16, 2023
(86) PCT No.: PCT/IB2023/058221

§ 371 Date: Jan. 29, 2025

(30) Foreign Application Priority Data
Aug. 31, 2022 (GB) 2212648.6

(51) Int. Cl. A62B 18/00; A61F 7/00; A62B 18/08; A62B 23/02; H04R 1/02; H04R 1/10

(52) U.S. Cl. CPC A62B 18/003; A61F 7/00; A62B 18/006; A62B 18/084; H04R 1/028; H04R 1/1008; H04R 1/105; A61F 2007/0005; A61F 2007/0067; A62B 23/02

(57) ABSTRACT

A wearable air purifier is provided and includes a headgear, an air delivery mask, and a fan. The air delivery mask is connected to the headgear and bounds an air delivery region. The air delivery mask is further configured to define a first airflow path to deliver a flow of air to the air delivery region. The headgear defines a second airflow path. The fan is configured to direct airflow to the first airflow path and the second airflow path.

### TECHNICAL FIELD

The present invention relates to a wearable air purifier and, more particularly but not exclusively, an air purifier which integrates audio headphone functionality.

### BACKGROUND

Air pollution is a well-known problem, particularly in urban areas and in public spaces where space between individuals may be limited, such as on public transport. A variety of air pollutants have known or suspected harmful effects on human health, such as toxic gases and other particulates such as dust and pollen.

The wearing of face masks is becoming more commonplace as individuals make positive steps to manage airborne contaminants. In its most basic form, a face mask may comprise one or more layers of fabric held over the mouth by an elastic strap. However, usually such face masks are passive and their filtering effectiveness is limited. More sophisticated approaches are now known which involve the use of a wearable air purifier, which provides a filtered airflow to the nose and mouth region of a user. An example of a wearable air purifier is disclosed in GB2595231A to Dyson Technology Limited.

The wearable air purifier in GB2592531A combines the functionality of a set of headphones with a mask or ‘visor’ which extends in front of a mouth region of a user to deliver a flow of purified air. One challenge with devices of this type is how to manage the thermal impact on the user wearing the device; prolonged use can increase temperature and cause perspiration around the ear region, for example. It is against this background that the invention has been devised.

### SUMMARY

In a first aspect, the examples of the invention provide a wearable air purifier, comprising a headgear, and an air delivery mask connected to the headgear and which bounds an air delivery region, wherein the air delivery mask is configured to define a first airflow path to deliver a flow of air to the air delivery region, wherein the headgear defines a second airflow path. The wearable air purifier conveniently further comprises a fan configured to direct airflow to the first airflow path and the second airflow path.

Beneficially, therefore, the wearable air purifier of the invention has a fan that that is configured to direct air to two separate flow paths, which provides a more space and weight efficient purifier product.

In some examples, the second airflow path may be defined in an ear piece path portion provided in at least one earpiece of the headgear. In such a configuration, the path portion may direct airflow into an ear chamber defined by the earpiece. This cools an ear of the user directly.

Alternatively, the path portion may direct airflow to an ear cushion of the earpiece, which cools the area of skin contact between an ear cushion or ear pad and the user.

In a further alternative, the headgear includes a headband which is supportable on the head of a user, and wherein the second airflow path is through a headband path portion. In this way, the airflow can also cool the head region of the user. The path portion may flow through a cushioned region, which provides a particularly convenient cooling mechanism. Perforations or apertures may be provided in the path portion to benefit airflow towards the head region.

In a second aspect, the examples of the invention provide a wearable air purifier, comprising a headgear, an air delivery mask connected to the headgear and which bounds an air delivery region, an airflow generator configured to direct airflow to the air delivery region through the air delivery mask, wherein the headgear defines an airflow path which is defined at least in part by a head-engaging surface. Beneficially, the airflow generator is configured to direct air through the airflow path to cool the head-engaging surface.

An advantage of the invention is that the wearable air purifier is configured to cool the head region of the user/wearer by way of the airflow that passes through the airflow path which is in part defined by the head-engaging surface. This provides enhanced comfort for the user during periods of prolonged wearing.

In one example, the head-engaging surface is a surface of a headband of the wearable air purifier. This directs the cooling effect specifically to the head of the wearer. More particularly, the airflow path may include a cushioned part of the headband. Foam-like material of the cushioned part may therefore defined at least in part the airflow path.

Alternatively, the head-engaging surface may be defined by a surface of an earpiece of the wearable air purifier, and particularly a cushioned ear pad of the earpiece. Cooling airflow paths may be provided in the ear piece as well as in the headband, to further enhance the cooling attributes of the device.

Apertures/perforations may be provided in the head-engaging surface to allow air to pass through, thereby further enhancing cooling.

In some examples, the air supply to the cooling airflow path and the air delivery mask can be selected by the user. This provides convenience for the user, as they can trigger the cooling airflow to operate independently to the purified airflow.

In a further aspect, the examples of the invention provide a headphone set comprising at least one earpiece defining a cushioned ear pad and including a speaker assembly for providing an audio output, and a headband for securing the headphone set to a head region of a user, wherein the ear pad and/or the head band defines at least in part an airflow path portion for conveying a flow of air, wherein the headphone set further comprises an airflow generator for generating a flow of air into the or each airflow path portion.

Features described above in connection with the first aspect of the invention are equally applicable to the second and/third aspect of the invention, and vice versa.

### BRIEF DESCRIPTION OF THE DRAWINGS

In order for the invention to be more fully understood, some specific implementations will now be described with reference to the following drawings, in which:

FIG. 1 is a front perspective view of an embodiment of a head wearable air purifier;

FIG. 2 is a front view of the head wearable air purifier of FIG. 1;

FIG. 3 is a perspective view of an air delivery mask of the wearable air purifier of FIGS. 1 and 2;

FIG. 4a is a side view of an earpiece of the wearable purifier of FIGS. 1 and 2, whereas FIG. 4b is a perspective view of the earpiece of FIG. 4a;

FIG. 5a is a cross-sectional view through the earpiece of FIG. 4a, and FIG. 5b is a further cross-sectional view through the earpiece of FIG. 5a to show a different viewing perspective;

FIG. 6 is a schematic view of the head wearable purifier shown in the previous Figures, and also illustrates an implementation of how cooling airflow may be incorporated into the purifier;

FIGS. 7 and 8 are further schematic views of the head wearable air purifier shown in the previous Figures, but shows further implementation examples of how cooling airflow may be incorporated into the purifier;

FIG. 9 is a small section of a headband of the air wearable air purifier;

FIGS. 10 and 11 are further schematic views of a wearable air purifier showing further implementation examples of how cooling airflow may be incorporated;

FIG. 12 is a schematic view of a control system that may apply to some examples of the wearable air purifier shown in the previous Figures.

### DETAILED DESCRIPTION

The term “air purifier” as used herein refers to a device or system capable of removing contaminants from air and emitting a supply of purified or filtered air. The term “head wearable” is used herein to define an item as being capable of or suitable for being worn on the head of a user. In a preferred arrangement, the head wearable air purifier comprises a headphone system comprising a pair of speaker assemblies mounted on a headband in which one or both of the speaker assemblies comprises an ear cup as described herein.

The term “headphones” as used herein refers to a pair of small loudspeakers, or speakers, joined by a headband that is designed to be worn on or around the head or neck of a user. Typically, the speakers are provided by electroacoustic transducers that convert an electrical signal to a corresponding sound. Circumaural headphones, often referred to as full-size or over-ear headphones, have ear pads whose shape is that of a closed loop (e.g. circular, elliptical etc.) so that they encompass the entire ear. Because these headphones completely surround the ear, circumaural headphones can be designed to fully seal against the head to attenuate external noise. Supra-aural headphones, often referred to as on-ear headphones, have ear pads that press against the ears, rather than around them. This type of headphone generally tends to be smaller and lighter than circumaural headphones, resulting in less attenuation of outside noise.

In this context, and in overview, the examples of the invention provide a wearable air purification product, system, device or item that is configured to improve the comfort of the product for the user, so that the user perceived a cooling effect in use, particularly in regions where the user comes into contact with the purifier, namely the headband and the ear cups. Various means are disclosed in the following discussion which result in a cooling effect for the user.

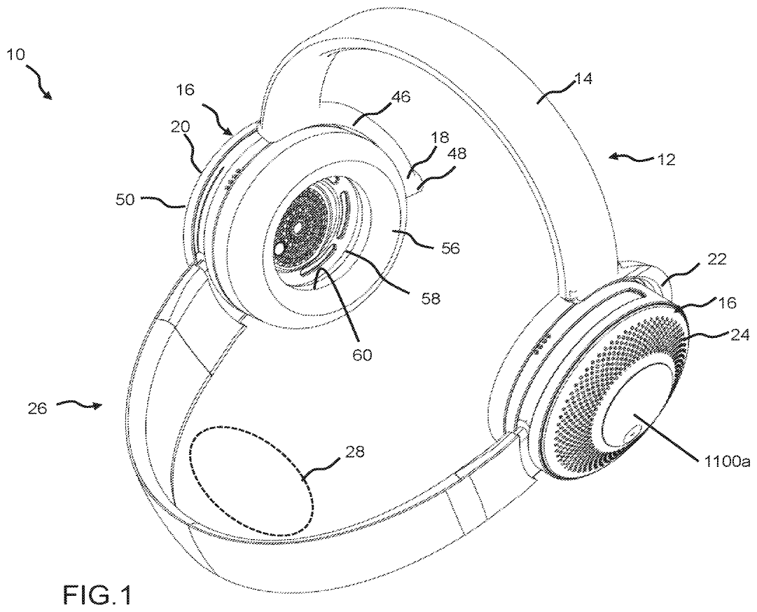

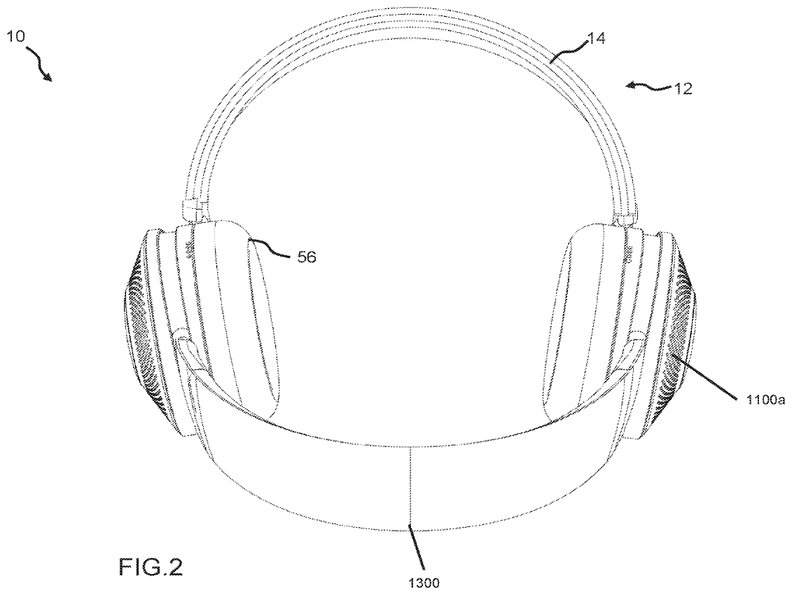

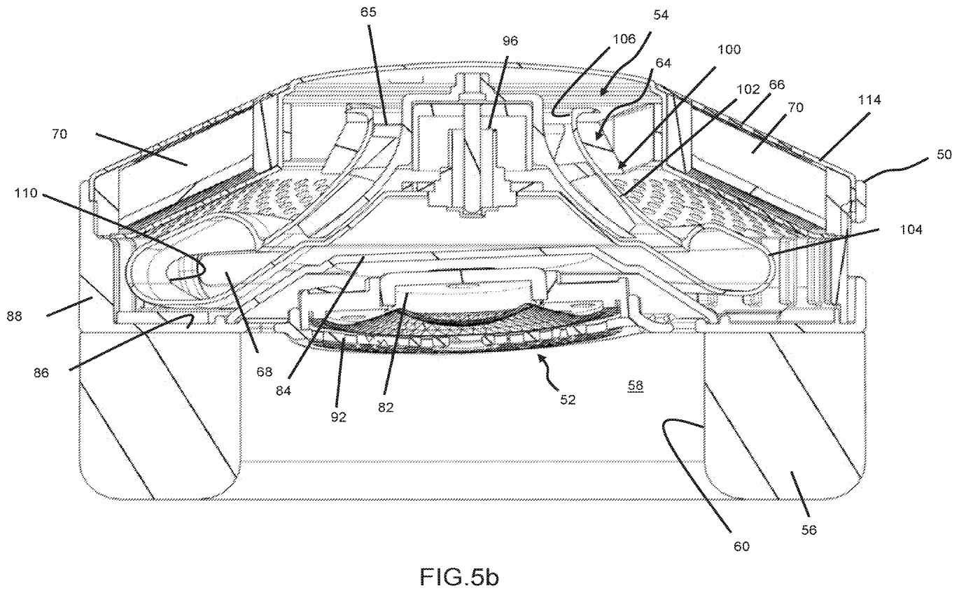

With reference to FIGS. 1 to 5, there is shown a wearable air purifier 10 to which the examples of the invention relate. The air purifier 10 shown here is very similar to devices disclosed in prior publications belonging to the applicant, such as GB2582372 and WO2020021231. A full discussion of the audio and air purification details will not be provided here for brevity, but a summary is provided below for completeness. It should be noted that FIG. 1 to 5b show an example of an air purification product to which the examples of the invention apply, as shown in the schematic views of FIG. 6 onward. Therefore, it should be noted that the wearable air purifier in FIGS. 1 to 5b provides a technical context into which the invention can be set.

The wearable air purifier 10 is configured to be worn on a user's head, like a conventional set of headphones. For this purpose, therefore, the wearable air purifier 10 comprises a headgear 12 comprising a wearable support or ‘headband’ 14 to which is connected a pair of audio earpieces or earcups 16. More specifically, a first end 18 of the headband 14 is connected to a first one 20 of the earpieces 16 and a second end 22 of the headband 14 is connected to a second one 24 of the earpieces 16.

The air purifier 10 also comprises a curved air delivery mask, nozzle or visor 26 that extends forward of the headband 14 and between the earpieces 16 in a pronounced curved configuration to resemble a chin-guard shape, thereby extending about to bound or encircle or define an air delivery region 28. In this example, the air delivery mask 26 is elongated and bar-like in form and is made from a rigid plastics material such as polycarbonate, although this is just optional. The air delivery mask 26 may be transparent, or at least translucent, in form which may be advantageous in certain respects.

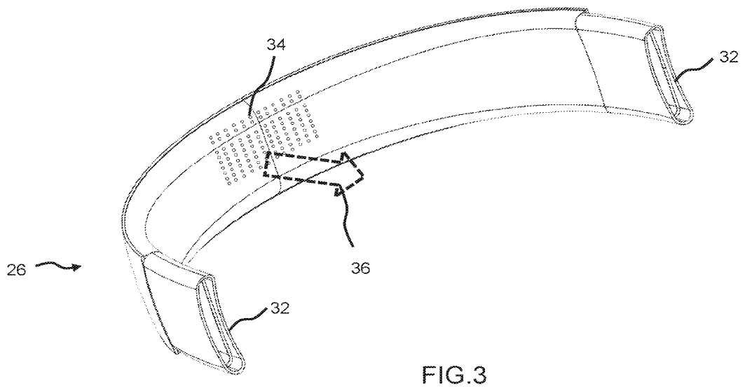

As seen in FIG. 3 more clearly, the air delivery mask 26 is generally a hollow structure and so includes suitable airflow pathways, ducts and channels 32 that convey a flow of air from the compressor along the air delivery mask 26 and direct it towards the user's mouth through at least one nozzle outlet 34. The at least one nozzle outlet 34 may be embodied by a grille or array of holes, as is appropriate. The flow of air is indicated here as ‘36’.

The headband 14 is coupled at its first and second ends 18,22 to respective ones 20,24 of the earpieces 16 by a conventional movable or rotatable coupling, embodied here by an arcuate arm 46 that extends about a portion of a respective earpiece 16 and engages with that earpiece 16 by a rotational pivot pin 48. It is to be noted that this method of coupling is disclosed here for completeness, and the skilled person would understand that other coupling schemes are possible and so further discussion of this coupling will be omitted for brevity.

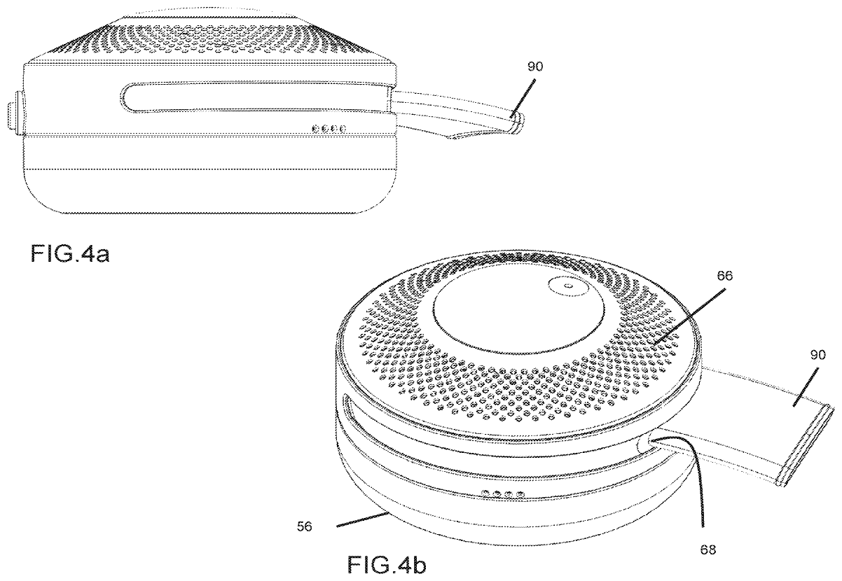

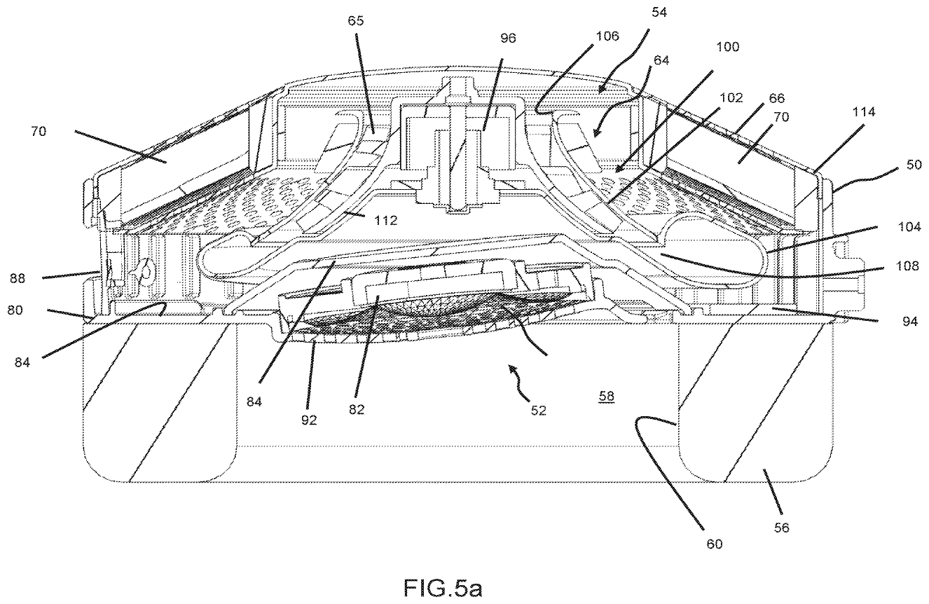

The pair of audio earpieces 16 are generally identical in this example, and cross sections through one the earpieces 16 are shown in FIGS. 5a and 5b. Reference will be made to a single earpiece in FIG. 4a/b and 5a/b, although it should be noted that the same general structure applies to both earpieces.

To provide dual audio and air flow functionality the earpiece 16 as shown comprises a housing or case 50 which encloses a speaker assembly 52 at a radial inward position and a compact air purifier unit 54 at a radially outer position. Here, the terms radially “inner” and “outer” are taken with respect to the head of a user on which the air purifier is worn. An ear pad 56 is provided to provide a soft cushioned interface for the user's ear. The housing 50 and the ear pad 56 together define a cavity 58 which has an opening 60. The opening 60 and the cavity 58 are shaped to accommodate a user's ear, in use.

The speaker assembly 52 includes a speaker unit 62 comprising speaker and speaker electronics (not shown). A Bluetooth® or other type of wireless communication transmitter/receiver may be provided for wireless communication with an audio playing device. The speaker unit 62 and the air purifier unit 54 may share a battery pack and part of the control electronics (not shown here).

The air purifier unit 54 comprises an airflow generator, compressor or fan assembly 64, in the form of a motor-driven impeller 65, located in the housing and which is arranged to create an airflow. The airflow generator 64 draws into the housing 50 through an air inlet 66 and expels the air flow from the housing 50 through an air outlet 68.

The airflow generator 64 includes a filter 70 through which airflow enters the housing 50. Therefore, the filter 70 is located downstream (i.e. relative to the airflow generated by the impeller) of the air inlet 66 of the housing 50 and upstream of the air outlet 68. In the illustrated embodiment, the filter 70 is also located upstream relative to the motor-driven impeller. Any suitable filter configuration may be used, such as a depth filter media or a surface filter media, such as pleased filter paper.

As will be appreciated from the discussion that follows, the airflow generator 64 is configured to define a space which accommodates at least a part of the speaker unit 62, which will now be described in more detail

In the illustrated embodiment, the housing 50 comprises a speaker chassis 80 upon which an acoustic driver unit 82 is mounted. A generally frusto-conical speaker cover 84 mounted on the speaker chassis 80 over the acoustic driver unit 82. The speaker chassis 80 comprises a generally circular base 86 that is surrounded by a cylindrical side wall 88. The air outlet 68 of the housing 50 is then defined by an aperture formed in the cylindrical side wall 88. The earpiece 16 is also provided with a hollow, rigid outlet duct 90 that extends from the housing 50 and that is arranged to connect the air outlet 68 of the earpiece 16 to couple to the inner duct 32 of the air delivery mask 26.

A central portion of the speaker chassis 80 provides a driver support plate 92 upon which the acoustic driver unit 82 can be located. The generally frusto-conical speaker cover 84 is then mounted on the speaker chassis 80 over the entirety of the driver support plate 92 such that the acoustic driver unit 82 is covered by the speaker cover 84. The driver support plate 92 of the speaker chassis 80 is provided with an array of apertures for allowing sound generated by the acoustic driver unit 82 to pass through the speaker chassis 80 into the cavity 58 enclosed by ear pad 56. In addition, the driver support plate 92 is angled or tilted relative to the peripheral portion of the base 84 of the speaker chassis 82. The angle or tilt of the driver support plate 92 is chosen so that the acoustic driver unit 82 is substantially parallel with the ears when the head wearable air purifier 10 is worn on the head of a user with the earpiece 16 over the user's ear.

Each of the earpieces 16 also comprises one or more circuit boards 94 upon which various electronic circuitry is disposed or mounted. For example, this electronic circuitry may comprise motor control circuitry that is arranged to control a rotational speed of a motor 96 that drives the airflow generator 54, audio control circuity that is arranged to control the audio playback and ANC circuitry that is arranged to implement active noise control to attenuate unwanted noise. In the illustrated embodiment, the one or more circuit boards 94 are disposed on or mounted to the peripheral portion of the speaker chassis 80. The circuit board 94 therefore at least partially encircles the acoustic driver unit 82 (i.e. is disposed outside/around a periphery of the acoustic driver unit 82) when the acoustic driver unit 82 is mounted on to the driver support plate 92.

A generally frusto-conical impeller casing 100 is mounted above the speaker cover 84, in the orientation of the drawings. The impeller casing 100 contains both the impeller 65 and the motor 96 and is disposed over the speaker cover 84 so that acoustic driver unit 82 is nested within a recess or cavity defined by a back/rear of the impeller casing 100. The impeller casing 100 comprises a generally frusto-conical impeller housing 102 surrounding the impeller 65 and the motor 94, and an annular scroll chamber or volute 104 fluidically connected to a base of the impeller housing 100 and that is arranged to receive the air exhausted from the impeller housing 100. The impeller housing 100 is provided with an air inlet 106 through which air can be drawn by the impeller 65 and an air outlet 108 through which the air is emitted from the impeller housing 100 into the annular volute 104. The air inlet 106 of the impeller housing 100 is provided by an aperture/opening at the small diameter end of the impeller housing 100 and the air outlet 108 is provided by an annular slot formed around a large diameter end or base of the impeller housing 100.

The annular volute 104 comprises a spiral (i.e. gradually widening) duct that is arranged to receive the air exhausted from the impeller housing 100 and to guide the air to an air outlet 110 of the volute 104. The air outlet 110 of the volute 104 discharges into an internal chamber 111 of the earpiece 19 which fluidically connects to the air outlet 68 of the housing 50. The term “volute” as used herein refers to a spiral funnel that receives the fluid being pumped by an impeller and increases in area as it approaches a discharge port. The air outlet 110 of the volute 104 therefore provides an efficient and quiet means for collecting the air that is exhausted from the circumferential annular slot that that forms the air outlet 110 of the impeller housing 100.

In the illustrated embodiment, the impeller 65 is a mixed flow impeller that has a generally conical or frusto-conical shape. The impeller 65 is hollow such that a rear/back side of the impeller 65 defines a generally frusto-conical recess. The motor 96 is then located within this recess. Preferably, the impeller 65 is a semi-open/semi-closed mixed flow impeller i.e. having a back shroud 112 only. The back shroud 112 of the impeller 65 defines the recess within which the motor 96 is nested/disposed.

The impeller casing 100 is supported suitably within the housing 50 by a plurality of resilient supports (not shown) that reduce the transmission of vibrations from the impeller casing 100. The resilient supports may suitably comprise an elastomeric material with an appropriate energy absorption characteristic.

To close off the outer upper surface (in the orientation of the drawings) of the earpiece 16, the housing 50 further comprises an outer cover 114 that is mounted onto the speaker chassis 80. This outer cover 114 is arranged to fit over (and therefore generally conforms to) the filter 70 and is provided with an array of apertures that allow air to pass through the outer cover 114 and that therefore define the air inlet 66 of the outer cover 114. These apertures are sized to prevent larger particles from passing through to the filter 70. Alternatively, in order to allow air to pass through, the outer cover 114 could comprise a grille or mesh mounted within windows in the outer cover 114.

The outer cover 114 may be releasably attached to the speaker chassis 80 so as to cover the filter 70. In this way, the outer cover 114 can be released to provide access to the underlying filter 70. Suitable attachment means can be provided for this purpose. Beneficially, the outer cover 114 protects the filter 70 from damage, for example during transit, and also provides a visually appealing outer surface in keeping with the overall appearance of the wearable purifier 10.

Returning to the overall configuration of the wearable purifier, as can be appreciated in FIG. 1, ends of the air delivery mask 26 are connected to the rigid outlet duct 90 that extends from the respective earpieces 16. In this way, the air delivery mask 16 is connectable to the earpieces and is configured to receive a flow of air from the airflow generator contained therein.

The air delivery mask 26 extends away from the earpieces 16 and assumes an arcuate shape so that both ends are suitably coupled to the respective earpieces 16. The air delivery mask 26 is arranged such that, when the purifier 10 is worn by a user with the first earpiece 20 over a first ear of the user and the second earpiece 24 over a second ear of the user, the air delivery mask 16 can extend around a face of the user, from one side to the other, and in front of a mouth of the user. In particular, the air delivery mask 26 extends around the jaw of the user, from adjacent to one cheek to adjacent the other cheek, without making contact with the mouth, nose or surrounding regions of the user's face. It is therefore useful that the at least a portion of the air delivery mask 26 is formed of a transparent or partially transparent material so that the user's mouth is visible, so not impairing communication.

In use, the wearable air purifier 10 is worn by a user with the earpieces 20,24 placed over respective ears and the air delivery mask 26 extending about the mouth region of the user. Within each earpiece 16, the rotation of the impeller 65 by the motor 96 will cause an airflow to be generated through the impeller casing 100 that draws air into the earpieces 16 through the air inlet 66 in the outer cover 114. This flow of air will then pass through the filter 70 thereby filtering and/or purifying the airflow. The resulting filtered airflow will then pass into the air impeller casing 102, whereupon the impeller 65 will force the filtered airflow out through the annular slot that provides the air outlet 110 of the impeller housing 100 and into the volute 108. The volute 108 then guides the filtered airflow through the air outlet 110 of the speaker assembly, through the rigid outlet duct 90 that extends from the earpiece 16, and into the hollow channel 32 defined by the air delivery mask 26.

The above discussion provides an overview of the structure and functionality of the wearable air purifier 10 shown in the Figures. One challenge associated with such a device is that a user may feel a level of discomfort due to perspiration, in use. This effects particularly, but not exclusively, the regions of skin contact with the device. For example, the primary areas of skin contact are along parts of the headband, but also at the earpieces.

The discussion with now focus on implementations of the wearable air purifier 10 in order to improve the comfort level for the user by providing cooling airflow.

FIG. 6 shows a first example implementation of a wearable air purifier 10 in schematic form in accordance with the invention. Since the form of the air purifier shown in FIG. 6 is generally the same as discussed above in relation to the previous Figures, the same reference numerals will be used to refer to common parts.

It will be appreciated that FIG. 6 demonstrates in schematic form an airflow path 120 that extends from each one of the earpieces 16, through the hollow interior of the air delivery mask 26, and out through the nozzle 34 of the air delivery mask to the air delivery region 28. Here, the airflow is fed from both earpieces 16 although in some examples, only one earpiece needs to feed the airflow. The air flow is shown by the arrows labelled F1.

However, in FIG. 6, it will be apparent that the wearable air purifier 10 has a second airflow path 122 which, as shown here, is provided at least in part in the headgear. The airflow path 120 running through the air delivery mask will be referred to as a first airflow path. In contrast, the airflow path 122 extending through the headgear will be referred to as a second airflow path.

Notably, the left hand earpiece 16 in FIG. 6 provides airflow to both the first airflow path 120 and the second airflow path 122. This is beneficial because it avoids the need of a dedicated fan to feed the second airflow path 122 which would otherwise increase the complexity, weight, and size of the overall package of the wearable air purifier, thereby increasing cost.

For the purposes of feeding the airflow into the second airflow path 122 from the earpiece 16, a suitable channel 124 is provided that bridges the earpiece 16 and the headband 14. The channel 124 may be formed as part of the structure that connects the earpiece 18 to the headband 14, which would provide a convenient and aesthetic solution. However, other options are possible, for example a separate pipe may be provided between the earpiece 16 and the headband 14. In this context, it should be noted that the channel 124 may be formed by a pipe or duct made from a suitably elastic material such a rubber. Such a material may help to damp vibrations from the compressor.

As can be seen in FIG. 6, the second airflow path 122 includes a path portion that extends along the headband 14 and is provided with at least one opening 126 to allow air to pass through. The flow of air can be appreciated by the arrows marked F2. The openings 126 are shown on the top side of the headband 14 in FIG. 6, but this is not essential and the openings 126 can be formed in any suitable part of the headband 14.

Beneficially, the air flowing along the second airflow path 122 provides a cooling effect for the user's head 130. The close proximity between the head 130 and the headband 14 is apparent in FIG. 6 with the result that the head 130 becomes warm when in contact with a head engaging surface 132 of the headband 14 for extended periods. The thermal energy generated by the head 130 will be transferred to the head engaging surface 132. However, since the airflow F2 is flowing past the head engaging surface 132, and since the head engaging surface 132 forms at least a part of the second airflow path 122, some of the thermal energy will be transferred to the airflow F2, thereby cooling the headband 14.

In the illustrated example in FIG. 6, the interior of the headband 14 is a cushioned region 133 being formed of a foamed material, having an open cell structure to allow the passage of air. This provides an cooling effect and cushioning for the headband 14. Therefore, in this example the second airflow path 122 is formed in a cushioned region of the headband 14.

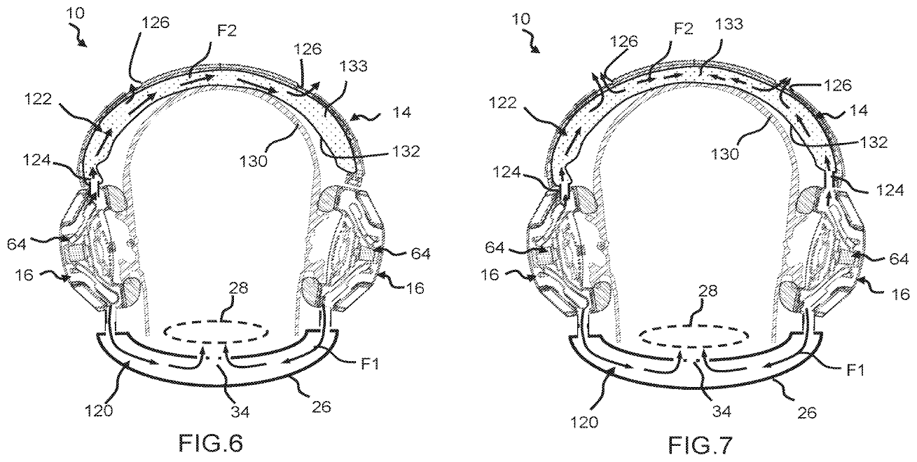

In the FIG. 6 example, the second airflow path 122 is fed airflow from the left hand earpiece. This may provide sufficient cooling airflow. However, in some examples, particularly where a higher flow of air is required, the second earpiece 16, that is the right hand earpiece as illustrated, could also contribute to the air flow in the second airflow path 122. This is shown in FIG. 7.

Both first and second earpieces 16 may contribute to the airflow generally equally. Alternatively, the contributions may be unequal. The airflow generators 64 may be controlled so as to operate together at the same general flow rate, or the airflow generators may be controlled to provided different levels of airflow based on a number of factors. In one envisaged example, sensing means (not shown) may be provided to sense the temperature at one or more points along the length of the headband 14, which data can be acted on by appropriate control algorithms to generate more or less flow as required.

It will be appreciated in the examples of FIGS. 6 and 7 that the head engaging surface is an unbroken surface so cooling relies on heat transfer across the surface. A mix of materials for the head engaging surface could be used in order to improve heat transfer. One example material would be for the headband 14 to comprise an open-celled foam material for the cushioning effect, which is covered with a rubberised material or fabric or an imitation leather style material for the head engaging surface 132.

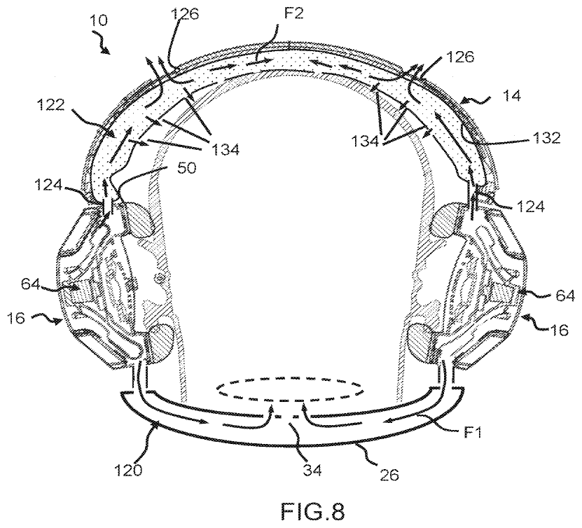

In another example, the head engaging surface 132 may be provided with one or more apertures to enable air to flow across, past or through the head engaging surface. The apertures may be few in number to enable a jet of air to emanate from the headband 14, albeit with that jet of air being at low pressure. Alternatively, there may be many apertures, for example as would be achieved with a perforated headband material. One such example is shown in FIG. 8.

It will be apparent that the example in FIG. 8 is comparable to FIG. 7, as it shows the second airflow path 122 extending through the headband 14. However, in addition, the head engaging surface 132 of the headband 14 is provided with a plurality of apertures 134.

The apertures 134 permit air to bleed out of the second airflow path 122 towards the head 130 of the user through the head engaging surface 132. The apertures 134 therefore provide an enhanced cooling effect.

At this point it should be noted that the apertures 134 in the underside surface of the headband 14 can be provided in addition or as an alternative to the apertures 126 provided in the upper surface of the headband 14.

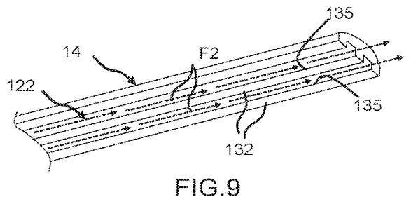

In principle, other structures to provide a cooling effect by way of the headband 14 are possible. For example, in FIGS. 6 to 8 the second airflow path 122 passes through the cushioning material of the headband. However, in an alternative example shown in FIG. 9, the second airflow path 122 is provided through a shallow channel, duct or passage 135 intermediate the headband cushioning and the head. In FIG. 9 it should be appreciated that only a short section of the headband 14 is shown to illustrate the principle. However, it can be seen that the headband includes a head engaging surface 132 that is downward facing and which includes two longitudinal channels 135. The channels 135 are in a parallel configuration and extend longitudinally along the headband 14, in this example.

The flow of air is shown by the dashed arrows marked F2, and it will be appreciated that this flow of air will serve to transport thermal energy away from the region of contact between the user's head and the headband 14. It should also be noted that the airflow path demonstrated by FIGS. 6 and 7, which can be considered ‘internal’ to the headband 14, could also be combined with external airflow channels like those shown in FIG. 9.

The examples shown in FIGS. 6 to 9 demonstrate how a first airflow path 120 is provided in the air delivery mask of the wearable air purifier device, and how a second airflow path 122 may be provided to generate a cooling effect at a region of contact between the user's head and a head engaging surface 132 of the device.

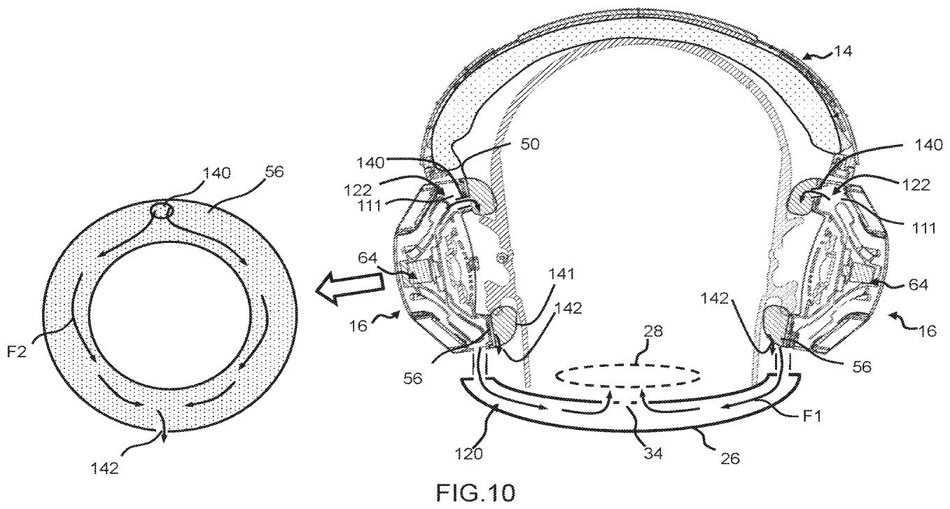

A further example is shown in FIG. 10. As will be appreciated from the similarity of the wearable air purifier 10 in Figure to that of FIGS. 6, 7 and 8, the device in FIG. 10 has a first airflow path 120 that is defined by the air delivery mask 26, and which is provided with airflow by the airflow generators 64 of each of the earpieces 16. The device also has a second airflow path 122 that serves to cool a region of contact between the user and the headgear.

However, rather than the second airflow path 122 being provided by the headband 14 of the device, the second airflow path 122 is defined in the earpieces 16. The earpieces therefore define respective portions of the second airflow path. As can be appreciated in FIG. 10, each earpiece 16 has an aperture 140 that provides that provides a route for air flow generated by the airflow generator 64 to pass from the air outlet 110/internal chamber 111 of the housing 50 into the ear pad 56.

FIG. 10 also shows the route that the airflow takes through the ear pad 56. The ear pad 56 therefore defines a portion of the second airflow path 122, and the outer surface of the ear pad 56 defines a head engaging surface 141 that is cooled by the airflow through the ear pad 56. As can be seen, in this example, the aperture 140 is positioned in the ear pad 56 near to the point where the earpiece 16 attaches to the headband 14. In principle, however, the aperture 140 may be positioned elsewhere. It is also envisaged that the surface 141 of the ear pad may be perforated or otherwise provided with one or more apertures where the ear pad is in contact with the user's head or ear to enhance the cooling effect.

Since the ear pads 56 are made from a foam material, the second airflow path is defined through the foam material itself due to the open-celled structure. Therefore, the air flows through the ear pad material from the aperture to an outlet 142. The outlet 142 is shown here as in a diametrically opposite position to the aperture 140 but this is not essential. The outlet 142 is defined singly in this example, but may instead include several apertures to provide an outlet for the airflow. Alternatively, all airflow may pass through the perforations in the ear pads 56 and the outlet 142 may be omitted.

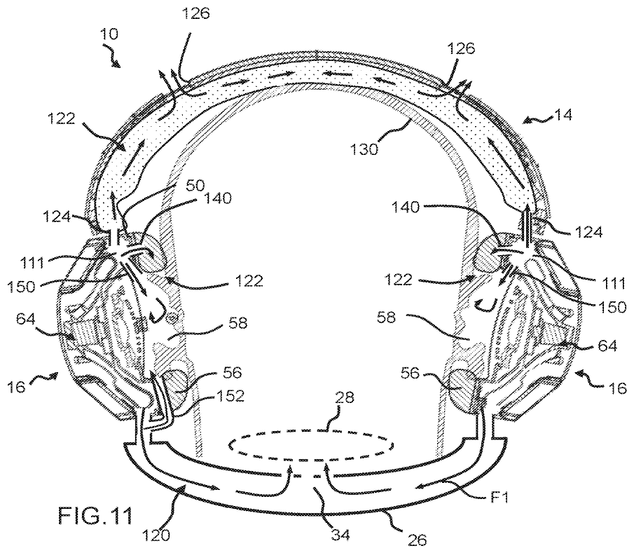

FIG. 11 shows a further example of where the airflow generators 64 in the earpieces 16 supply a cooling flow of air to the wearable air purifier 10. As in the previous figures, the wearable air purifier 10 has a first airflow path 120 that is provided to the air delivery mask 26. That airflow is generated by the airflow generators 64 in the earpieces 16.

Further flows of air are directed into the headband 14, and also into the earpads 56. The configuration of the headgear to enable the flow of air into the headband 14 and the ear pads 56 can be considered the same as described above, combining the examples in FIGS. 8 and 10. In FIG. 11 however, both additional airflows are provided.

Furthermore, the wearable air purifier 10 is configured to provide a further cooling airflow to the earpieces 16, in addition to the airflow that is directed into the ear pads 56. As is shown, an aperture 150 is provided in each of the earpieces 16 which fluidly connects the air outlet 110/internal chamber 111 of the housing 50 into the cavity 58 of the earpieces 16. The second airflow 122 therefore goes into the cavity 58 and cools the user's ear directly.

In this illustrated example, cooling airflows are provided in the headband 14, into the earpads 56 and also into the cavities 58. However, it is to be noted that the three separate cooling airflows are shown for completeness, and each may be embodied separately.

FIG. 11 also shows an alternative air flow route into the cavity 58, that can be provided instead or in addition to the apertures 150 in the respective earpieces 16. In this case, an aperture 152 is provided which extends from a portion of the air delivery mask 26 to the cavity 58. The aperture 152 is formed by a pipe or tube like structure that extends from a portion of the air delivery mask 26 that is near to where the mask joins onto the earpiece 16. The aperture 152 extends from a part of the housing 50 of the earpiece 16 or may extend through a part of the ear cup 56. In this example, therefore, the airflow into the cavity 58 is taken as a bleed off the first airflow path 120 along the air delivery mask 120. It is to be noted that the further aperture 152 is provided only for one of the earpieces 16 in the illustrated example, but that it may be provided for both earpieces 16.

In the case where there is more than one second airflow path 122 provided, for example as shown in FIG. 11, the wearable air purifier 10 may be configured to control the flow of air through each of the air paths, and optionally also the rate of airflow. Moreover, in the case where there is a single second airflow path 122, the wearable air purifier device may be configured to control the rate of the airflow.

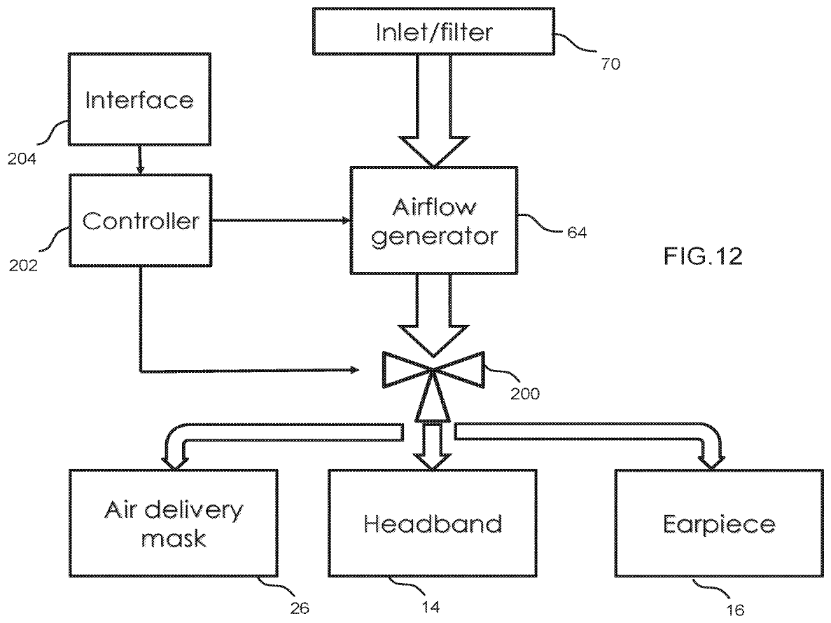

Therefore, as discussed above, in all configurations of the wearable air purifier 10 shown here, the airflow generator 64 may be operable to control the airflow through the second airflow path 122, whether it is formed in the headband 14 or the earpieces 16. This is depicted in FIG. 12, in which the airflow generator 64 is shown as receiving a flow of air from the filter 70 and providing a flow of air towards the air delivery mask 26, the headband 14 and the earpiece 16. To this end a control valve 200 is provided as part of a control system of the wearable air purifier device and therefore is operably connected to a controller 202.

The controller 202 is operatively connected to the airflow generator 64 and the control valve 200, in this example. Therefore, the controller 202 can control the flow rate and also control the destination of the air flow. The precise form of the control valve 200 is not important. However, the control valve 200 should have the functionality required to supply a flow of air to one or more of the air delivery mask 26, the headband 14 and the earpieces 16. One such valve may be a three-way modulating micro spool valve.

The control over the control valve 200 can be in response to various factors. One such factor may be a control input from a user on a user interface 204. The user interface 204 may take various forms. For example, it may be a touch screen that provide a visual display with which the user may interact. However, to be helpful for a user to operate without sight, it may be the case that the user interface is embodied as one or more tactile switches, and/or a multiple position switch. The user interface 204 may also be responsive to voice commands from the user.

The controller 202 is used here as a general term to refer to the computing hardware, software and firmware required to control the electronic functionality of the wearable air purifier device. In overview, the controller 202 may include suitable modules (not shown) to control various aspects of the wearable air purifier device, and may comprise an audio control module, an active noise cancelling module, a wireless module, a microphone module, a battery and power management module, a sensor and data interface module, an antenna module, a fan control module and a valve control module. Moreover, the controller 202 is provides with a suitable processing environment, memory capacity and communications capability to enable it to carry out its functions.

Various modifications to the illustrated examples have been mentioned in the above discussion. The skilled person would, however, realise that other modifications may be made without departing from the invention as defined in the claims.

In the above aspects of the invention, the airflow generator 64 is provided in the earpiece of the wearable air purifier device. However, it should be noted that the invention is not limited to this configuration and that, as such the airflow generator may be provided external to the earpiece whilst still delivering the advantages of the illustrated examples of the invention.

1. A wearable air purifier, comprising:
a headgear,
an air delivery mask connected to the headgear and which bounds an air delivery region, and
a fan,
wherein the air delivery mask is configured to define a first airflow path to deliver a flow of air to the air delivery region,
wherein the headgear defines a second airflow path, and
wherein the fan is configured to direct airflow to the first airflow path and the second airflow path.

2. The wearable air purifier of claim 1, wherein the headgear includes at least one earpiece, and wherein the second airflow path includes an earpiece path portion defined by the at least one earpiece.

3. The wearable air purifier of claim 2, wherein the earpiece path portion is configured to direct airflow into an ear chamber defined by the at least one earpiece.

4. The wearable air purifier of claim 2, wherein the earpiece path portion is configured to direct airflow to an ear cushion of the earpiece.

5. The wearable air purifier of claim 2, wherein the at least one earpiece includes a speaker assembly.

6. The wearable air purifier of claim 1, wherein the headgear includes a headband wearable on or around a head region of a user, and wherein the second airflow path includes a headband path portion defined by the headband.

7. The wearable air purifier of claim 6, wherein the headband path portion includes a cushioned region.

8. The wearable air purifier of claim 6, wherein the headband path portion defines one or more apertures to allow air to flow towards the head region of a user.

9. The wearable air purifier of claim 8, wherein the one or more apertures are provided at least in part by a perforated surface.

10. The wearable air purifier of claim 6, wherein the headband defines one or more air outlets in an upper surface thereof configured to permit airflow to discharge from the second airflow path.
